(19) United States
(12) Patent Application Publication
(10) Pub. No.: US 2013/0184752 A1
(43) Pub. Date: Jul. 18, 2013

(54) SPINOUS PROCESS FUSION DEVICE

(71) Applicant: Binder Biomedical, Inc., Boca Raton, FL (US)

(72) Inventor: Lawrence J. Binder, Miami, FL (US)

(73) Assignee: Binder Biomedical, Inc., Boca Raton, FL (US)

(21) Appl. No.: 13/726,689

(22) Filed: Dec. 26, 2012

Related U.S. Application Data
(60) Provisional application No. 61/580,395, filed on Dec. 27, 2011.

(51) Int. Cl. A61B 17/70

(52) U.S. Cl. CPC A61B 17/7068
USPC 606/248

(57) ABSTRACT

A spinous process device has a first plate and a second plate that are connected by an arm. The first and second plates have contact surfaces that face each other, so a spinous process can be clamped between the contact surfaces. The second plate is adjustable, so the angle and distance of the second plate relative to the first plate can be varied and set as desired.

The Current Application depends from, and claims priority to, U.S. Provisional Patent Application No. 61/580,395, which was filed on Dec. 27, 2011.

### BACKGROUND OF THE INVENTION

a. Field of the Invention

The invention relates to the field of spinal surgery, and more particularly to a spinous process fusion device with plates connected by a joining arm.

b. Background of the Invention

The central nervous system is part of an overall system that functions to coordinate human activity. It is made up of the brain and the spinal cord. The main function of the spinal cord is to act as a conduit to communicate neuronal signals from the brain to the rest of the body. Protecting the spinal cord is the spinal column, or commonly referred to as the spine or vertebral column. Anatomically, the spinal column is made up of several regions, including the cervical, thoracic, lumbar and sacral regions. The cervical spine is made up of 7 seven vertebrae and functions to support the weight of the head. The thoracic spine is made up of 12 vertebrae and functions to protect the organs located within the chest. Five vertebrae make up the lumbar spine. The lumbar spine contains the largest vertebra and function as the main weight bearing portion of the spine. Located at the base of the spine is the five fused vertebrae known as the sacrum. The coccyx sits at the base of the spinal column and consists of four fused vertebrae.

Each of the vertebrae associated with the various spinal cord regions are made up of a vertebral body, a posterior arch, a spinous process, and transverse processes. The vertebral body, often described as having a drum-like shape, is designed to bear weight and withstand compression or loading. In between the vertebral bodies are intervertebral discs. The discs help cushion the spine against various movements and can be the source of various diseases. The posterior arch of the vertebrae is made up of the lamina, pedicles and facet joints. Transverse processes extend outwardly from the vertebrae and provide the means for muscle and ligament attachment, which aid in movement and stabilization of the vertebra.

One of the more common ailments associated with the spinal cord is damage to the spinal discs. Damage to the discs results from physical injury, disease, genetic disposition, or as part of the natural aging process. Disc damage often results in intervertebral spacing not being maintained, causing pinching of exiting nerve roots between the discs, resulting in pain. For example, disc herniation is a condition in which the disc material bulges from the disc space between the two vertebrae bodies. It is the bulging of the disc material which causes impingement on the nerves, manifesting in pain to the patient. In the treatment of severe cases, or in cases which have developed into spinal instability, the posterior elements such as the spinous processes are distracted, the impingement on the spinal cord is addressed and the spinous processes are locked in place with plates and/or screws.

### SUMMARY OF THE INVENTION

The instant invention describes an implantable surgical device designed for insertion between bone structures, particularly two spinous processes, thus preventing or limiting the motion of said involved vertebrae. The spinous process device has a first and second plate connected by an arm. The position of the second plate can be adjusted relative to the arm, so the first and second plates can be secured to different sized vertebra.

### BRIEF DESCRIPTION OF THE FIGURES

FIGS. 1-15 illustrate one embodiment of the design. FIG. 1 is an exploded axial view of this one embodiment.

FIG. 2 is an exploded lateral view.

FIG. 3 is an exploded perspective view.

FIG. 4 is an axial view.

FIG. 5 is a top view.

FIG. 6 is a lateral view.

FIG. 7 is a perspective view.

FIG. 8 is a top view illustrating adjustability of the mechanism.

FIG. 9 is an axial view illustrating adjustability of the mechanism.

FIG. 10 is a perspective view illustrating adjustability of the mechanism.

FIG. 11 is a top view illustrating adjustability of the mechanism.

FIG. 12 is an axial view illustrating adjustability of the mechanism.

FIG. 13 is a cross-sectional top view illustrating the interface between the components and the adjustability of the mechanism.

FIG. 14 is a top view illustrating the screw mechanism actuated bringing one plate closer to the other.

FIG. 15 is an axial view illustrating the screw mechanism actuated bringing one plate closer to the other.

FIGS. 16-32 illustrate another embodiment of the design.

FIG. 16 is an exploded axial view of the mechanism.

FIG. 17 is an exploded top view of the mechanism.

FIG. 18 is an exploded perspective view of the mechanism.

FIG. 19 is an axial view of the mechanism.

FIG. 20 is a top view of the mechanism.

FIG. 21 is a perspective view of the mechanism.

FIG. 22 is a top view illustrating the adjustability of the mechanism.

FIG. 23 is an axial view illustrating the adjustability of the mechanism.

FIG. 24 is a perspective view illustrating the adjustability of the mechanism.

FIG. 25 is an axial view illustrating the adjustability of the mechanism.

FIG. 26 is a lateral view.

FIG. 27 is a cross-sectional top view illustrating the adjustability of the mechanism.

FIG. 28 is a cross-sectional lateral view illustrating the locking mechanism.

FIG. 29 is a lateral view illustrating the device implanted between two spinous processes.

FIG. 30 is an axial view illustrating the device implanted between two spinous processes.

FIG. 31 is a posterior view of the spine and top view of the device illustrating the device implanted between two spinous processes.

FIG. 32 is a perspective view illustrating the device implanted between two spinous processes.

### DETAILED DESCRIPTION OF THE INVENTION

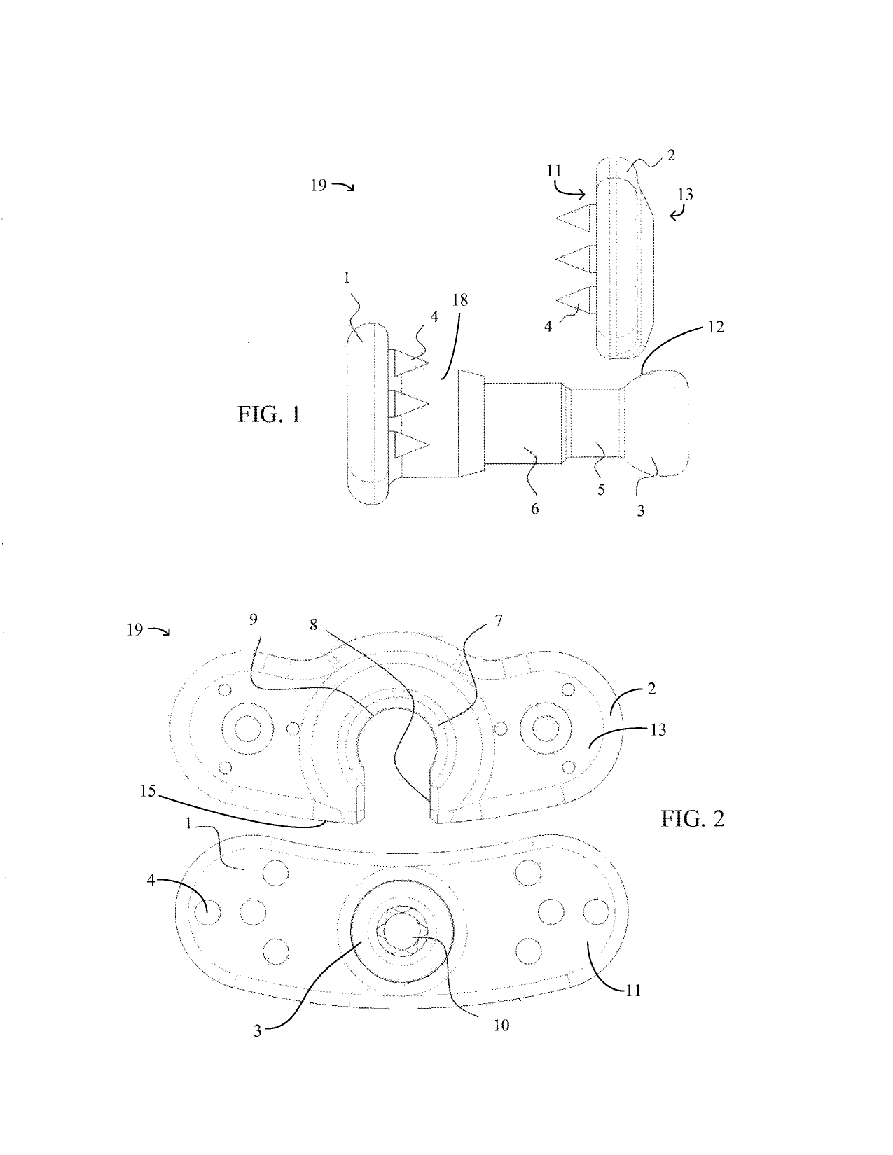

40 FIG. 1 illustrates a spinous process device 19 with a first plate 1 and a second plate 2 with spikes 4 and linearly adjustable extended arm 5. The arm 5 has a head 3 at the distal end of arm 5. A threaded socket 18 extends from the first plate 1, and a threaded region 6 of the arm 5 engages and is screwed into the threaded socket 18 of the first plate 1. The arm 5 can be connected to the first plate 1 by the threaded socket 18 and the threaded region 6. The head 3 has angled sides 12, so a cross section of the head 3 at the point where the head 3 connects to the arm 5 is smaller than a cross section of the head 3 at a point further away from the first plate 1. The angled sides 12 means the head 3 tapers.

FIG. 2 illustrates the pocket 7 that accepts the head 3 and allows adjustment between the plates 1, 2. It also illustrates the relief area 8 designed for ease of assembly, where the relief area 8 is a hole extending from an edge 15 of the second plate 2 to the pocket 7. The pocket 7 comprises an interference region 9 which is always smaller that the diameter of the head 3, therefore pulling the two plates 1, 2 together upon actuation. The interference region 9 is sized and shaped to match the angled sides 12 of the head 3, so the main shaft of the arm 5 passes through the relief area 8 and into the pocket 7, but the wider portions of the head 3 are too large to pass through the relief area 8 or the pocket 7. Therefore, the head 3 is locked into the pocket 7. When the head 3 is seated in the pocket 7, the second plate 2 and the arm 5 form a ball and socket type connection, which provides for a ball and socket type motion. A ball and socket motion is motion around an indefinite number of axis with have a common center. A drive receptacle 10 is positioned in the head 3, and in particular at the distal end of the head 3. A driver (not shown) can be inserted into the drive receptacle 10 and twisted to screw the threaded region 6 into or out of the threaded socket 18, thereby adjusting the distance between the first and second plates 1, 2.

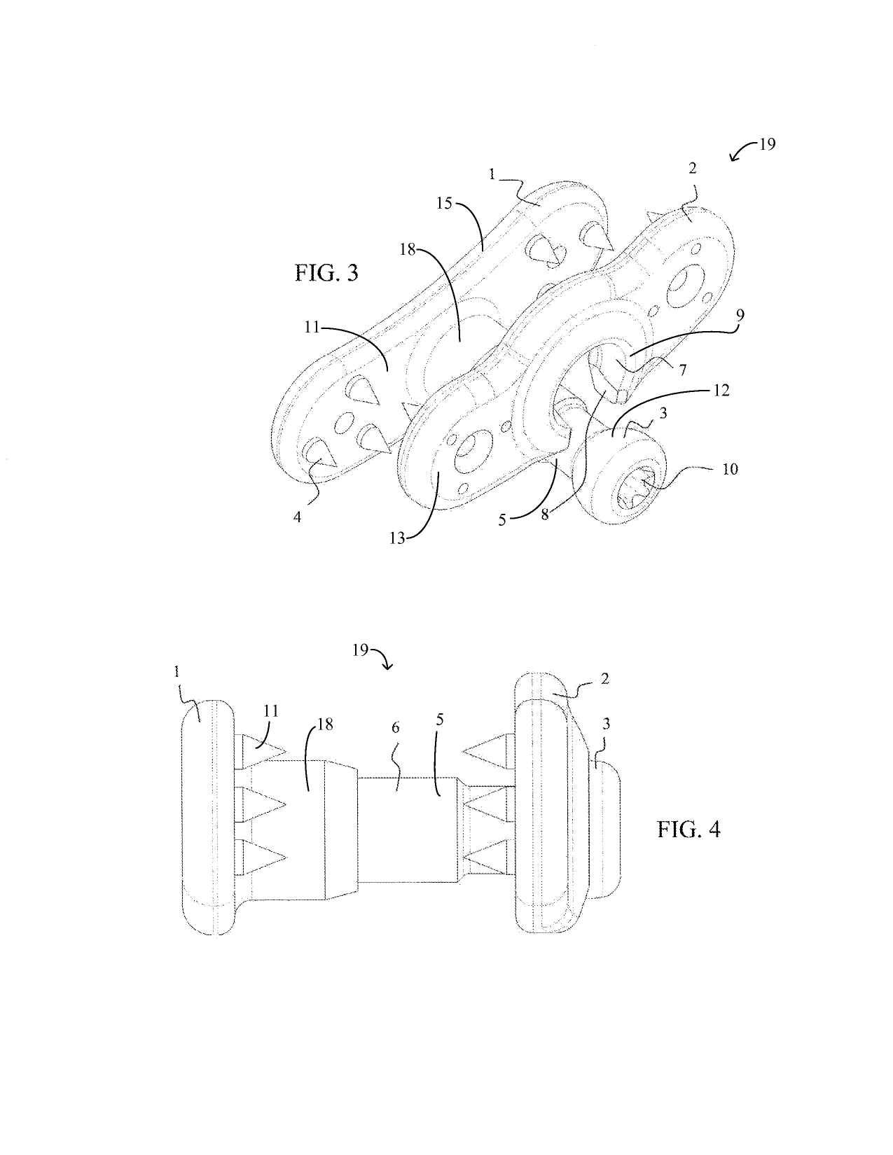

FIG. 3 illustrates a pocket 7 on the second plate 2 and a mating head 3 on the distal end of the extended arm 5. A contact surface 11 is seen on the first plate 1, where the contact surface 11 is the surface facing the second plate 2. The second plate has a corresponding contact surface 11 facing the first plate 1, but the second plate contact surface 11 is not visible in this drawing. An outer surface 13 of the second plate 2 is visible, where the outer surface 13 faces away from the first plate 1. The first plate 1 also has an outer surface 13 facing away from the second plate 2, but the first plate outer surface 13 is not visible in this drawing. The contact surface 11 has texture to produce a frictional surface. In this embodiment, the texture is spikes 4, but the texture could be other shapes, such as ridges, waves, dimples, or other shapes.

FIG. 4 illustrates the threaded region 6 of the extended arm 5 screwed into the threaded socket 18 of the first plate 1. The distal end of the head 3 is seen extending beyond the second plate 2, but the distal end of the head 3 could be flush or counter sunk in the second plate in other embodiments. In this embodiment, the arm 5 is rigidly fixed to the first plate 1, and the arm 5 is perpendicular to the first plate 1 as well.

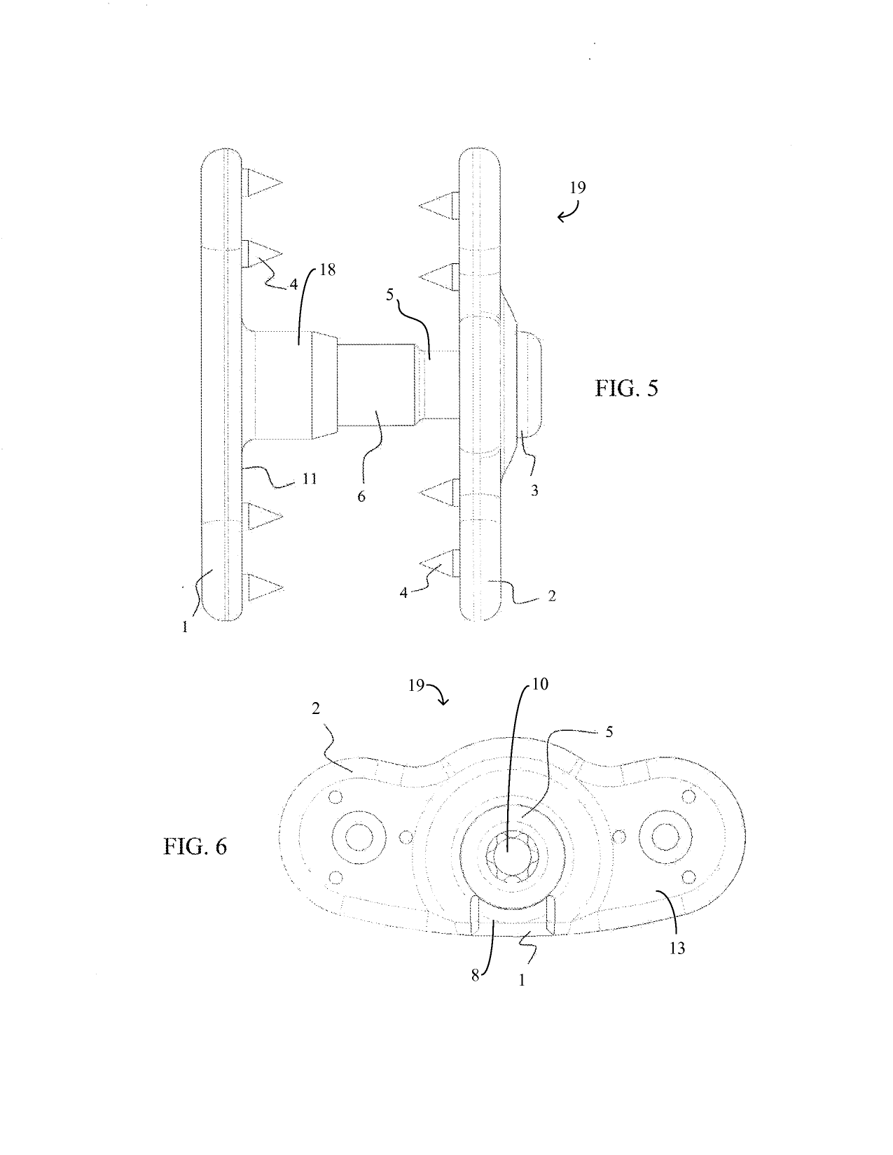

FIG. 5 illustrates the plates 1, 2 the threaded region 6 of the extended arm 3 screwed into the threaded socket 18, and the spikes 4 on the contact surface of both the first and second plates 1, 2.

FIG. 6 illustrates the second plate 2, and a small portion of the first plate 3 is visible in the relief area 8 of the second plate 2. The distal most surface 5 of the arm 5 is also visible, as well as the drive receptacle 10 in the head 5.

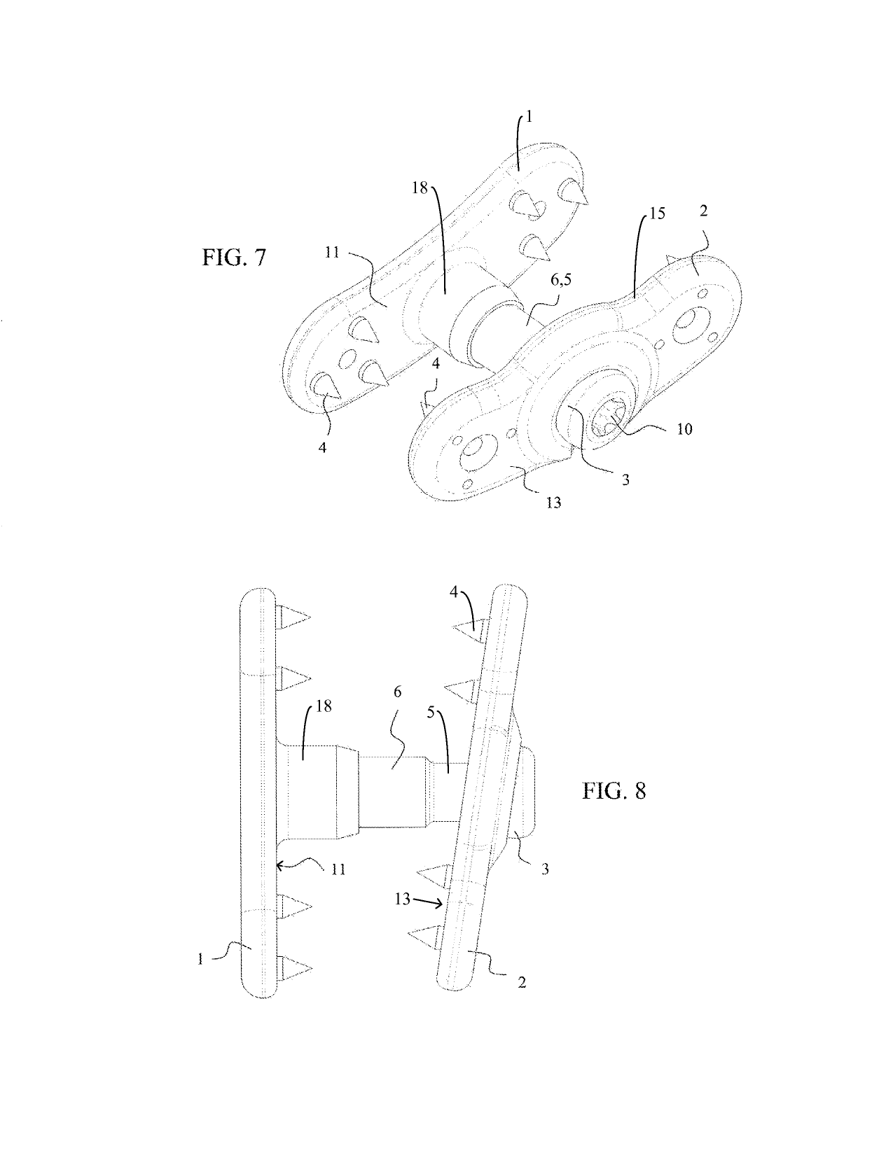

FIG. 7 illustrates the plates 1, 2, the threaded region 6 of the extended arm screwed into the threaded socket 18, the spikes 4 and the drive receptacle 10 for the arm. It also shows the spinous process contact surface 11 of the first plate and the outer surface 13 of the second plate. The second plate 2 is angled relative to the first plate 1, and the round nature of the head 5 and the pocket 7 allow the second plate 2 to rotate about the head and become angled relative to the first plate 1. The threaded arm allows for adjustment of the distance between the first and second plates 1, 2, so the combination of an arm 5 with an adjustable distance and ball and socket type connection between the arm 5 and the second plate 2 allows for a wide degree of adjustability for the spinous process device 19. The articulation of the second plate 2 about the arm 5 means the second plate contact surface 11 and the arm 5 can form a wide variety of angles, from less than 90 degrees to over 90 degrees, and the angles can be adjusted or manipulated as desired.

FIG. 8 illustrates the ability for the first and second plates 1, 2 to rotate in the top plane, but the design of the head 5 and pocket 7 allow the second plate 2 to rotate about the head 3 in the axial plane as well. The second plate 2 could also rotate about the head 3 so the first and second plates 1, 2, were not aligned, so the round cross section of the head 3 and the pocket 7 allow for motion in any direction, as long as the head is seated in the pocket 7.

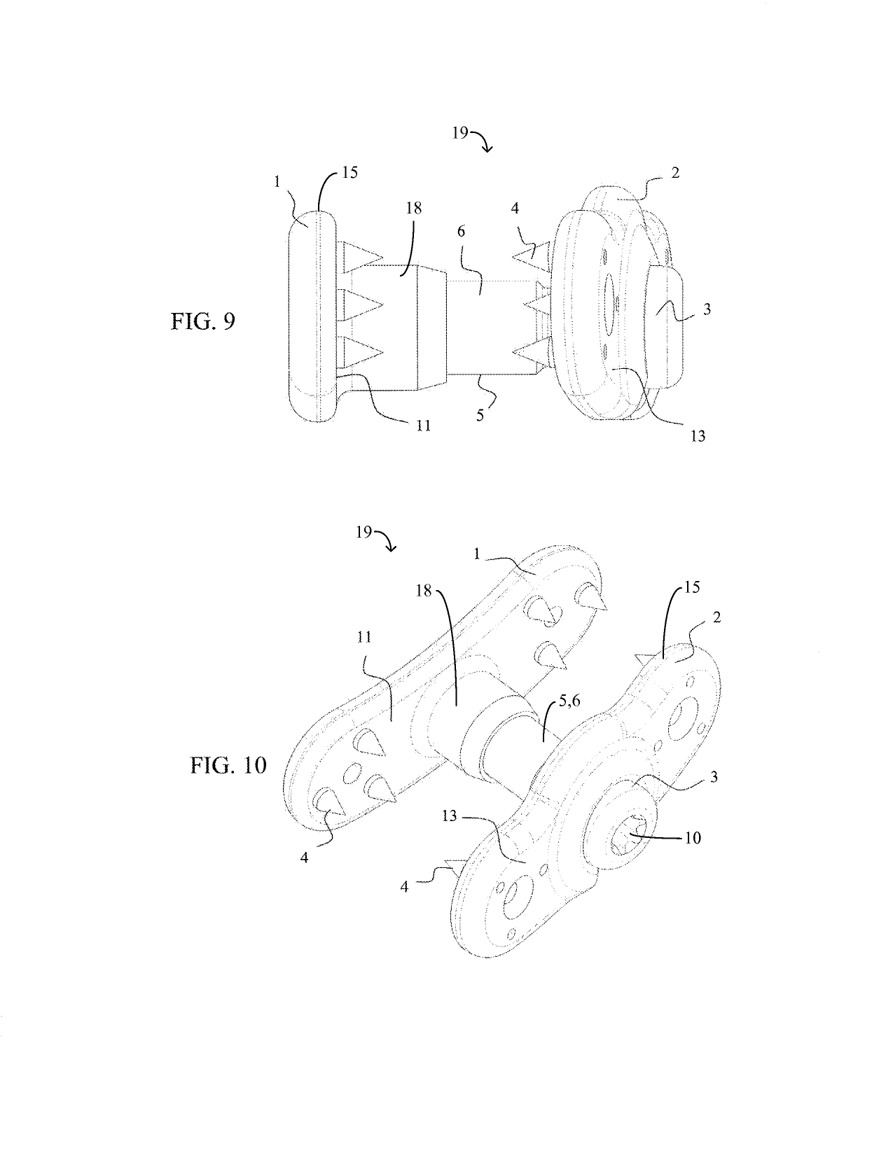

FIG. 9 illustrates the ability for the first and second plates 1, 2 to rotate, bend, and angle in the top plane.

FIG. 10 illustrates the ability for first and second plates 1, 2 to rotate, bend, and angle in the top plane.

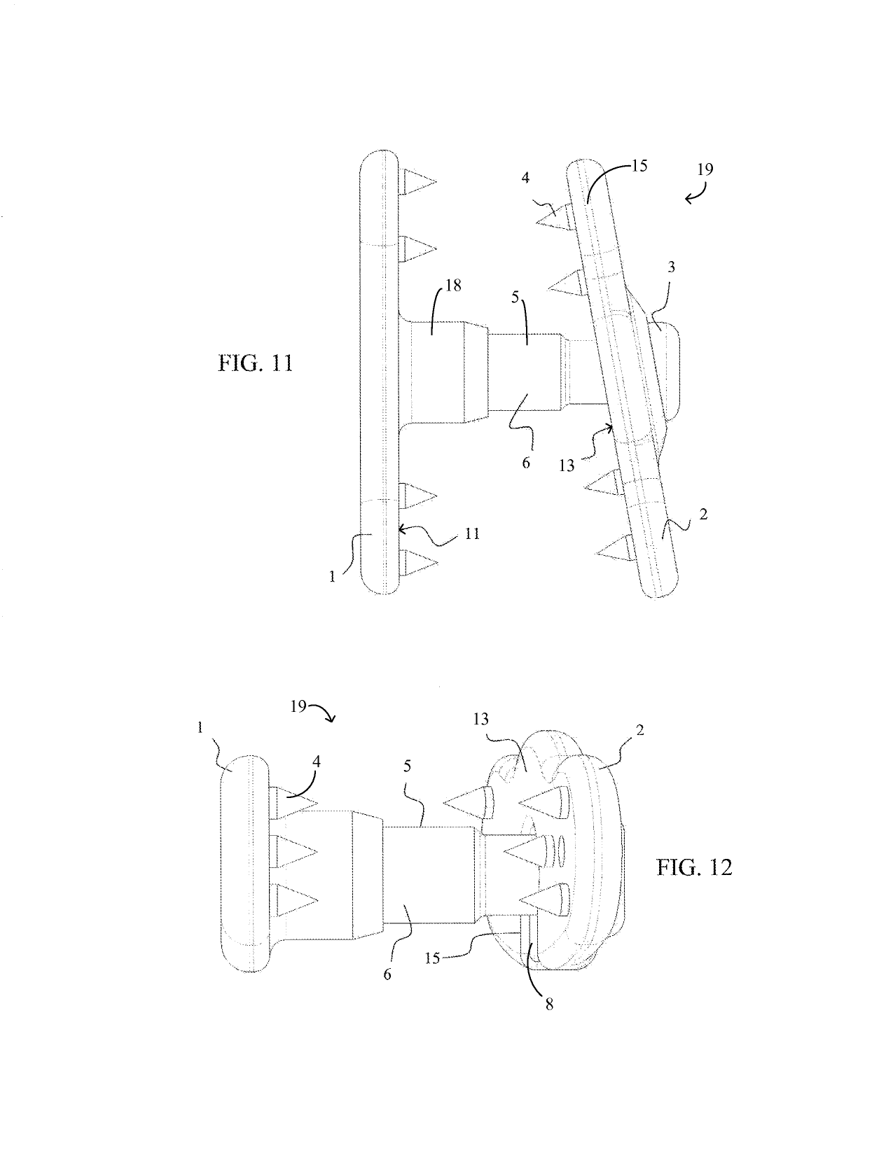

FIG. 11 illustrates the ability for the first and second plates 1, 2 to rotate, bend, and angle in the top plane.

FIG. 12 illustrates the ability for the first and second plates 1, 2 to rotate, bend, and angle in the top plane. It can also angle in the axial plane where the angle created by arm 5 and the second plate contact surface 11 can be greater or less than 90 degrees.

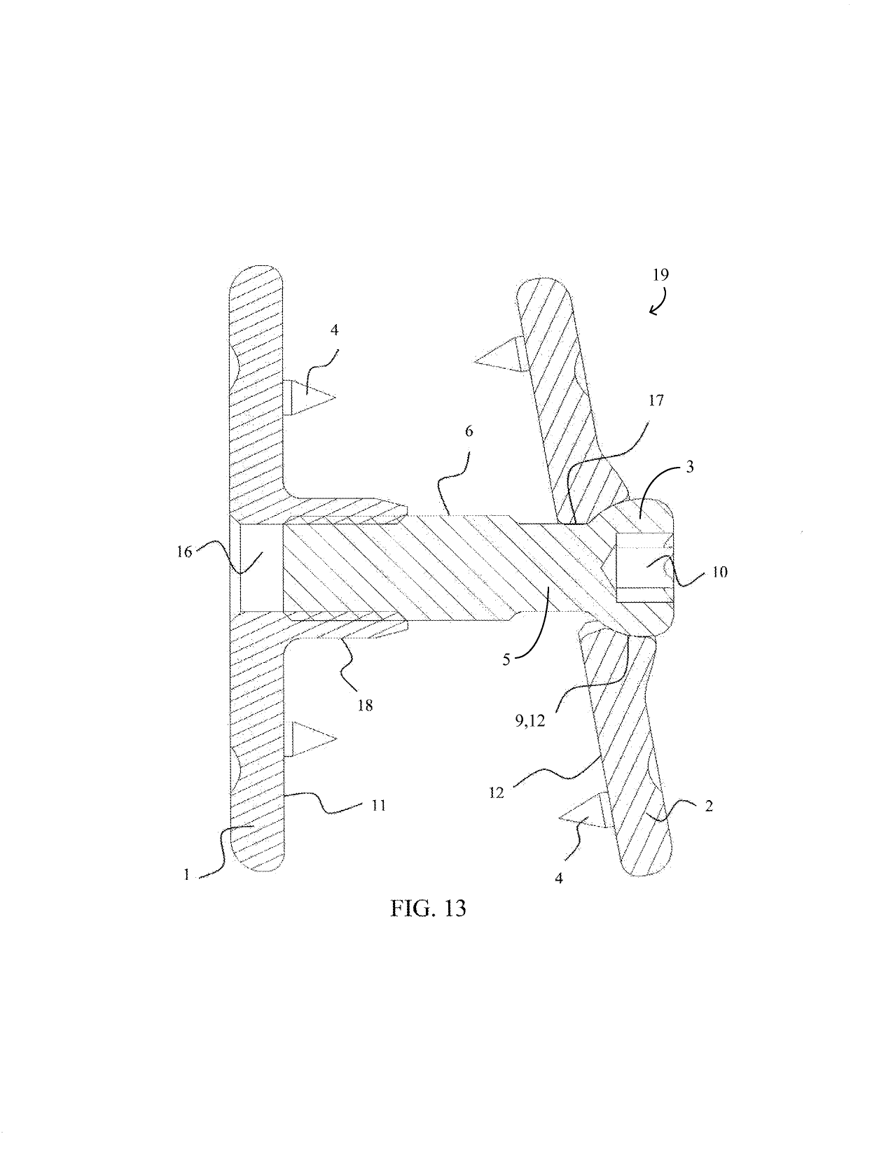

FIG. 13 illustrates the ability for the first and second plates 1, 2 to rotate about the contact area between the angled sides 12 of the head 5 and the interference region 9 of the pocket 7. The threaded region 6 of the arm 5 is also shown within the threaded socket 18, where the threaded socket 18 is a unitary part of the first plate 1. The threaded socket 18 could also be a separate component connected to the first plate 1 in other embodiments. Twisting the arm 5 by use of the drive receptacle 10 will screw the arm 5 either further into the threaded socket 18 or further out of the threaded socket 18, so the threaded gap 16 will become larger, or smaller, and the distance between the first and second plate 1, 2, can be adjusted. There can also be a stop surface 17 as part of the pocket 7, where the stop surface 17 limits plate angulation for the second plate 2 relative to the arm 5.

FIG. 14 illustrates the ability for the first and second plates 1, 2 to move closer to each other, thereby clamping material or vertebra between the contact surfaces 11 of the first and second plates 1, 2. In this embodiment, the threaded region 6 of the arm 5 is visible beyond the outer surface 13 of the first plate 1. However, in other embodiments, there can be a wall to prevent the threaded region 6 from extending beyond the outer surface 13 of the first plate 1.

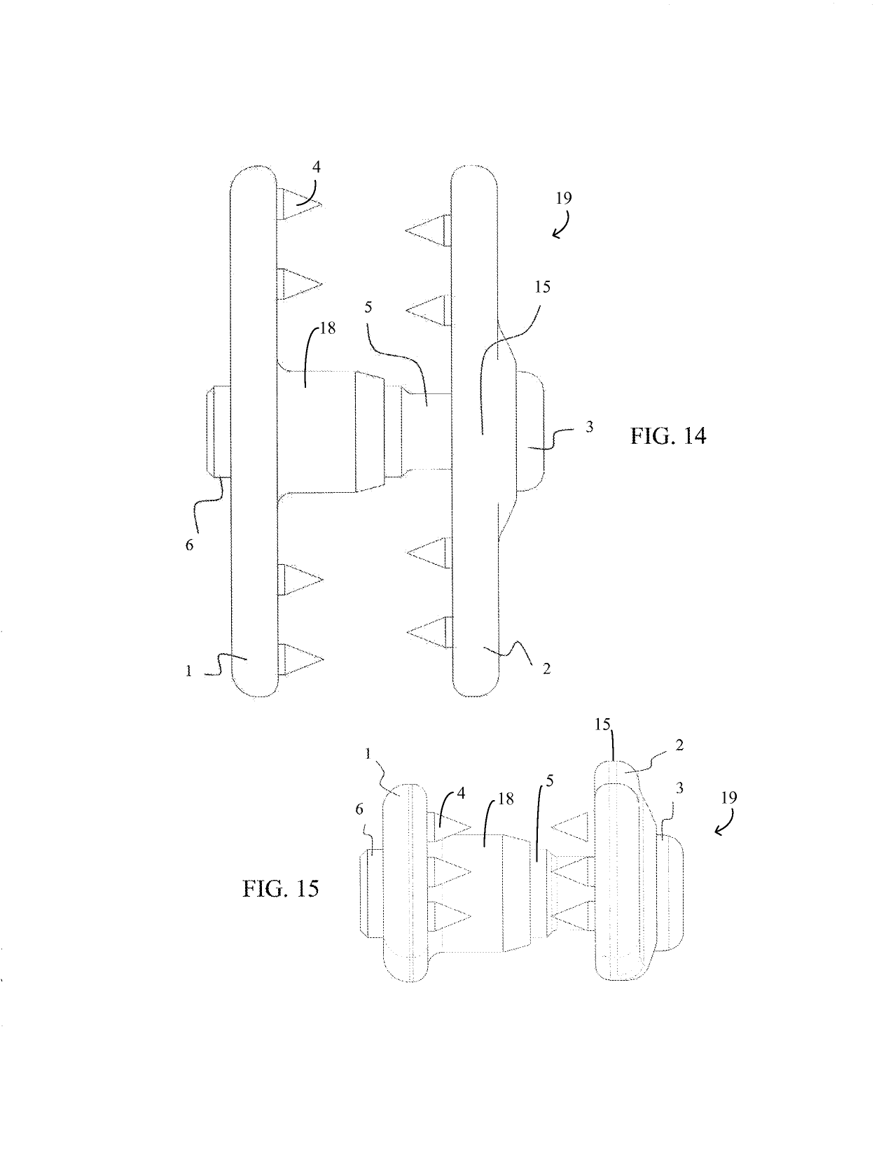

FIG. 15 illustrates the ability for the first and second plates 1, 2 to move closer to each other to clamp spinous processes or other vertebra components between the first and second plates 1, 2. The spikes 4 tend to drive into the vertebra between the first and second plates 1, 2 to secure the vertebra in place. The spikes 4 therefore provide texture and a frictional surface to keep the spinous process device 19 from slipping out of place.

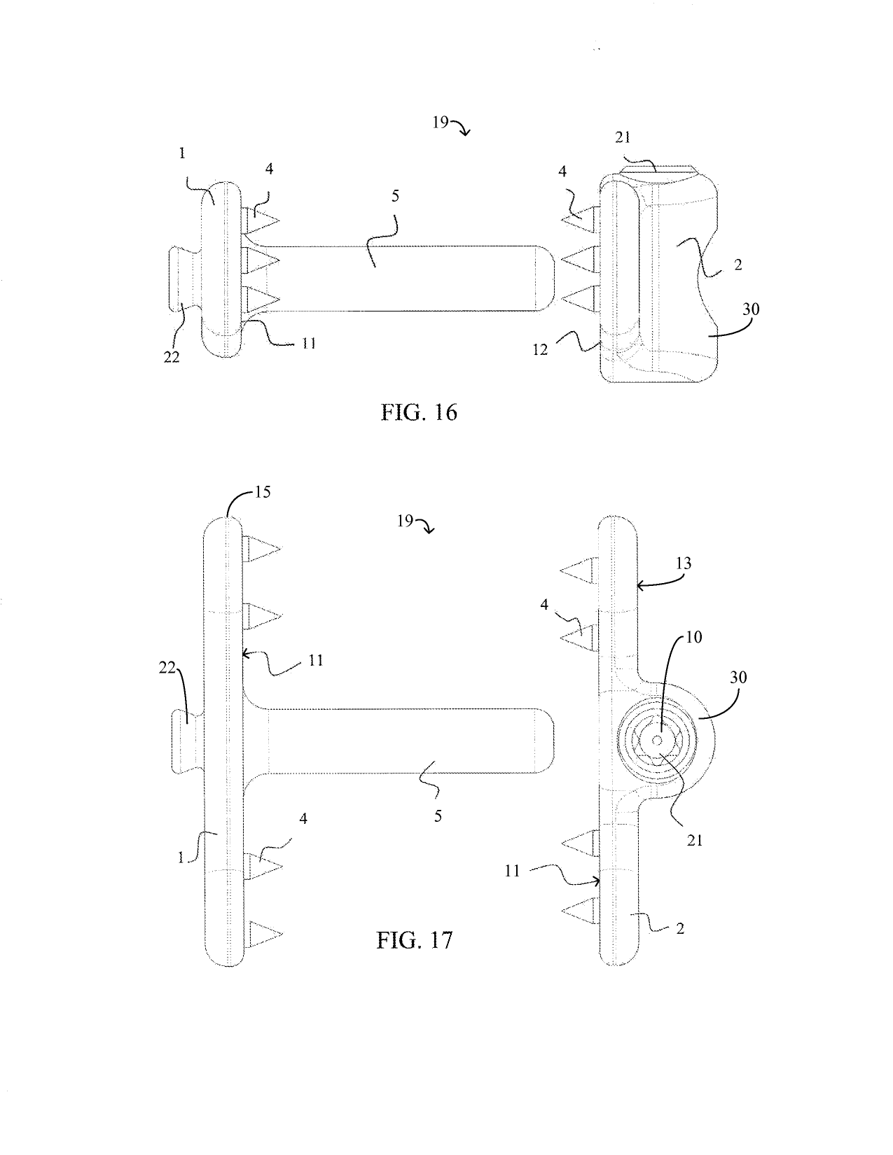

FIG. 16 illustrates the first plate 1 with a solid cylindrical extended arm 5 and the second plate 2 with a housing 30 for a cavity 26, where a set screw 21 penetrates the housing 30 so the set screw can be threaded into the arm 5 when the arm is within the cavity 26. A holding tab 22 for placing the spinous process device 19 is also shown here.

FIG. 17 illustrates the first plate 1 with a cylindrical arm 5 and the second plate 2 with a set screw 21. The set screw 21 comprises a drive receptacle 10, and a drive can be used to thread the set screw 21 into or out of the cavity housing 30. The drive can be a hex key or Allen wrench, a screw driver, or a wide variety of other devices that can be used to rotate a set screw.

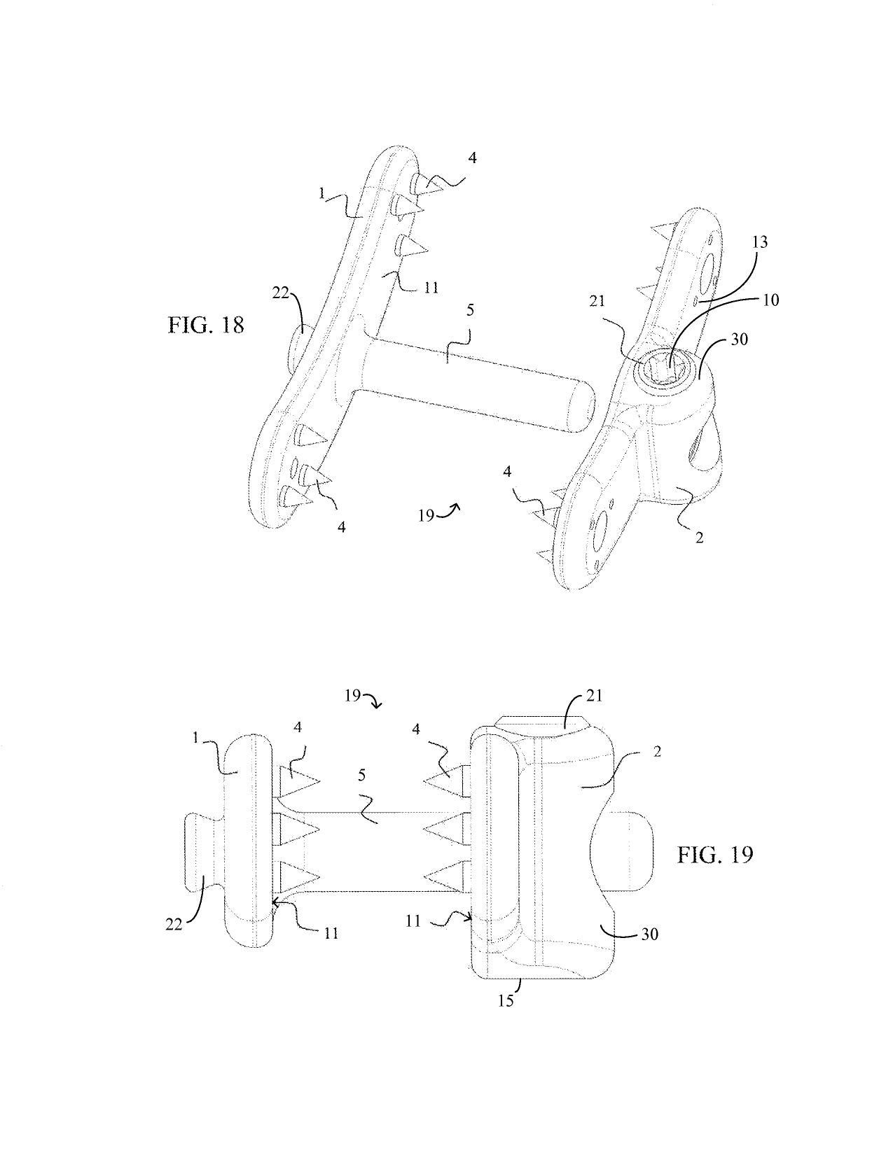

FIG. 18 illustrates the first plate 1 with the arm 5 and the second plate 2 with a set screw 21 and a housing 30 for the cavity 26. The arm 5 can pass into the cavity 26 to a point where the set screw 21 can contact the arm 5, and in some embodiments the arm 5 can pass completely through the cavity 26 and extend beyond the outer surface 13 of the second plate 2 and the housing 30.

FIG. 19 illustrates the first plate 1 with the arm 5 extending through the housing 30 and the second plate 2, with set screw 21.

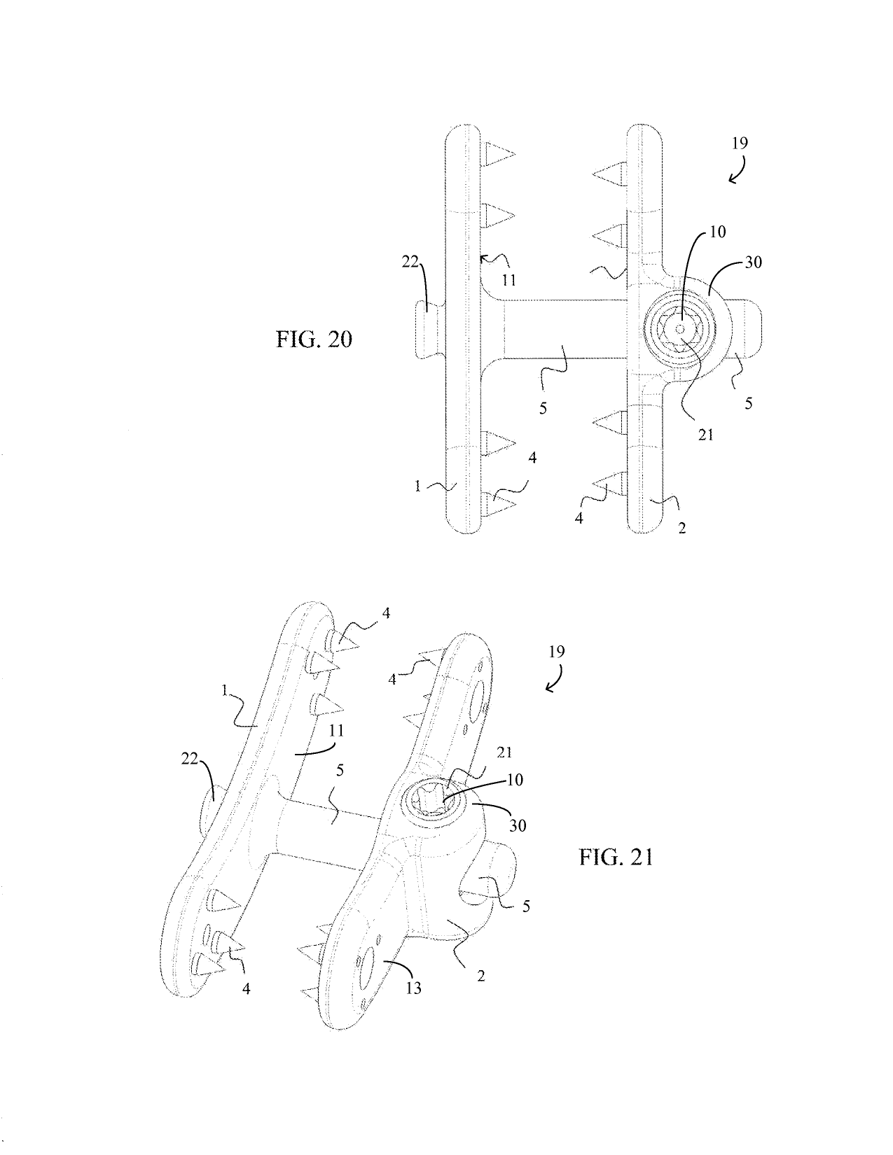

FIG. 20 illustrates the first plate 1 with the arm 5 and the second plate 2 with the set screw 21.

FIG. 21 illustrates a different view of the first plate 1 with the arm 5 and the second plate 2 with the set screw 21.

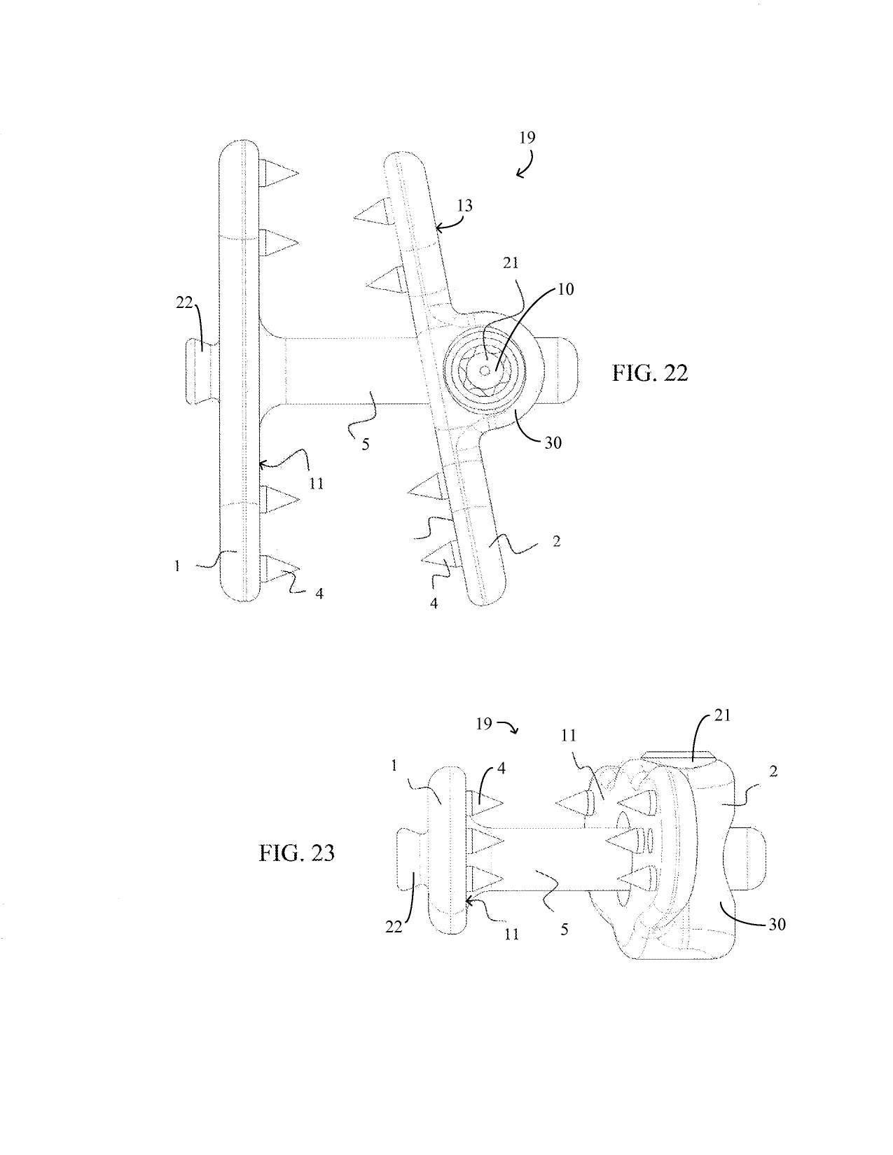

FIG. 22 illustrates the ability for the first and second plates 1, 2 to angle in the top plane. As can be seen, the contact surface 11 of the second plate 2 forms an angle with the arm 5 that is not a ninety degree angle.

FIG. 23 illustrates the ability for the first and second plates 1, 2 to angle in the top plane.

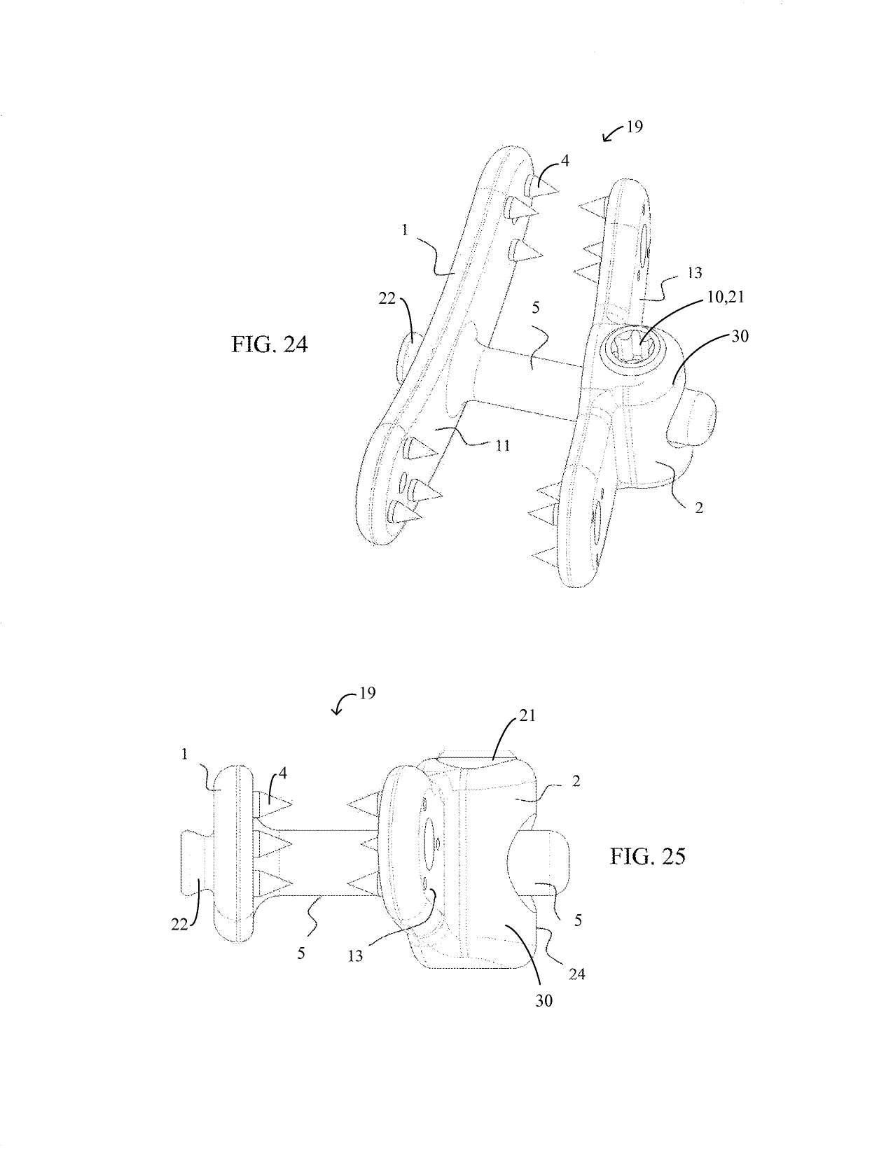

FIG. 24 illustrates a different angle showing the ability for the first and second plates 1, 2 to angle in the top plane.

FIG. 25 illustrates the ability for the first and second plates 1, 2 to angle in the top plane. It can also angle in the axial plane where the angle created by the arm 5 and the contact surface 11 of the second plate 2 can be greater or less than 90 degrees.

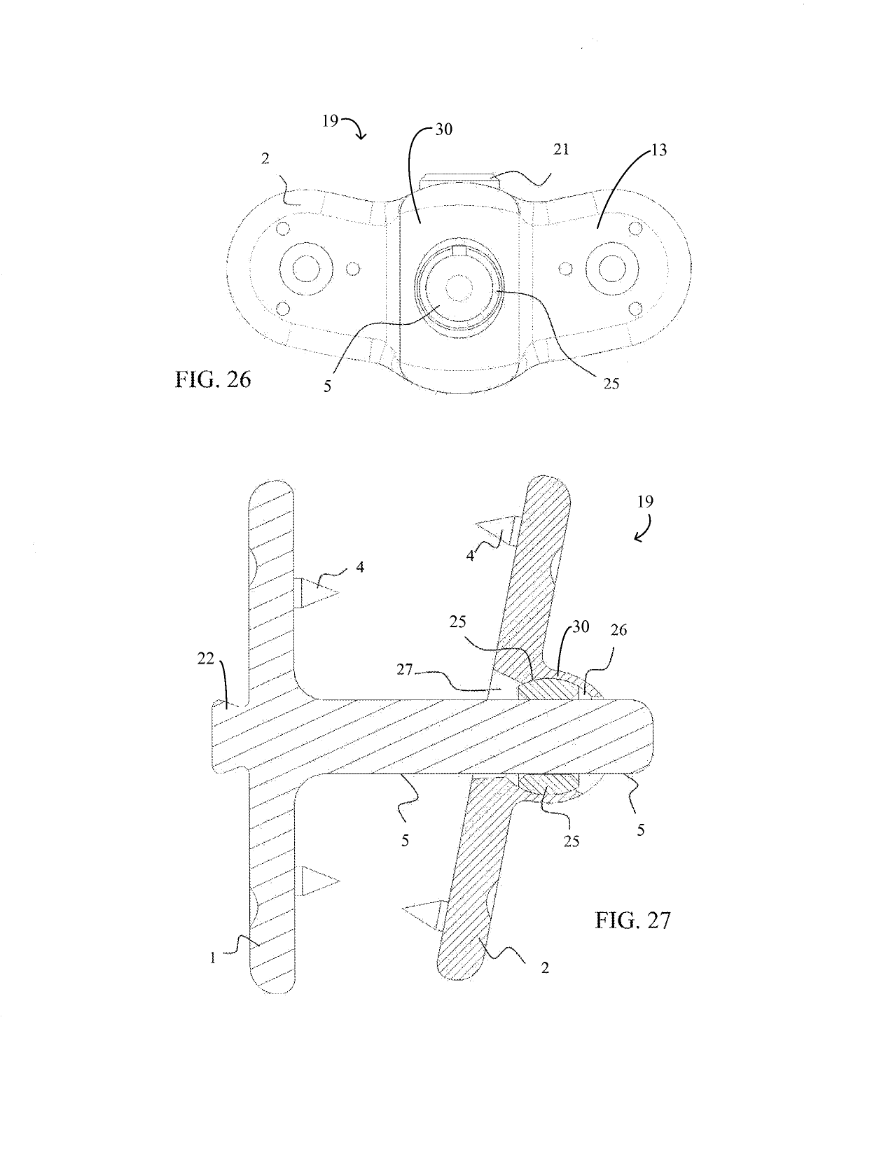

FIG. 26 illustrates the second plate 2, a bushing 25 positioned within the cavity in the housing 30, and the set screw 21.

FIG. 27 illustrates how the mechanism allows the second plate 2 to rotate and angle about the arm 5. The bushing 25 is positioned in the cavity 26 within the housing 30, and the cavity 26 can have a spherical shape that is matched by the outer surfaces of the busing 25. The inner surfaces of the bushing 25 can match the outer surface of the arm 5. The spherical cavity 26 and bushing 25 allow the second plate 2 to move in many different directions about the arm 5, in a ball and socket type of motion. An angulation cone 27 can be defined in the second plate 2 at the bottom of the cavity 26, where the angulation cone 27 extends outward from the cavity. The sides of the angulation cone 27 contact the arm 5 and limit the range of motion of the second plate 2 relative to the arm 5. The distance between the first and second plates 1, 2 can be adjusted by sliding the second plate 2 up or down on the arm 5 before locking the second plate 2 in position by tightening the set screw. The arm 5 can be a unitary part of the first plate 1, or the arm 5 can be a separate component that is attached to the first plate 1.

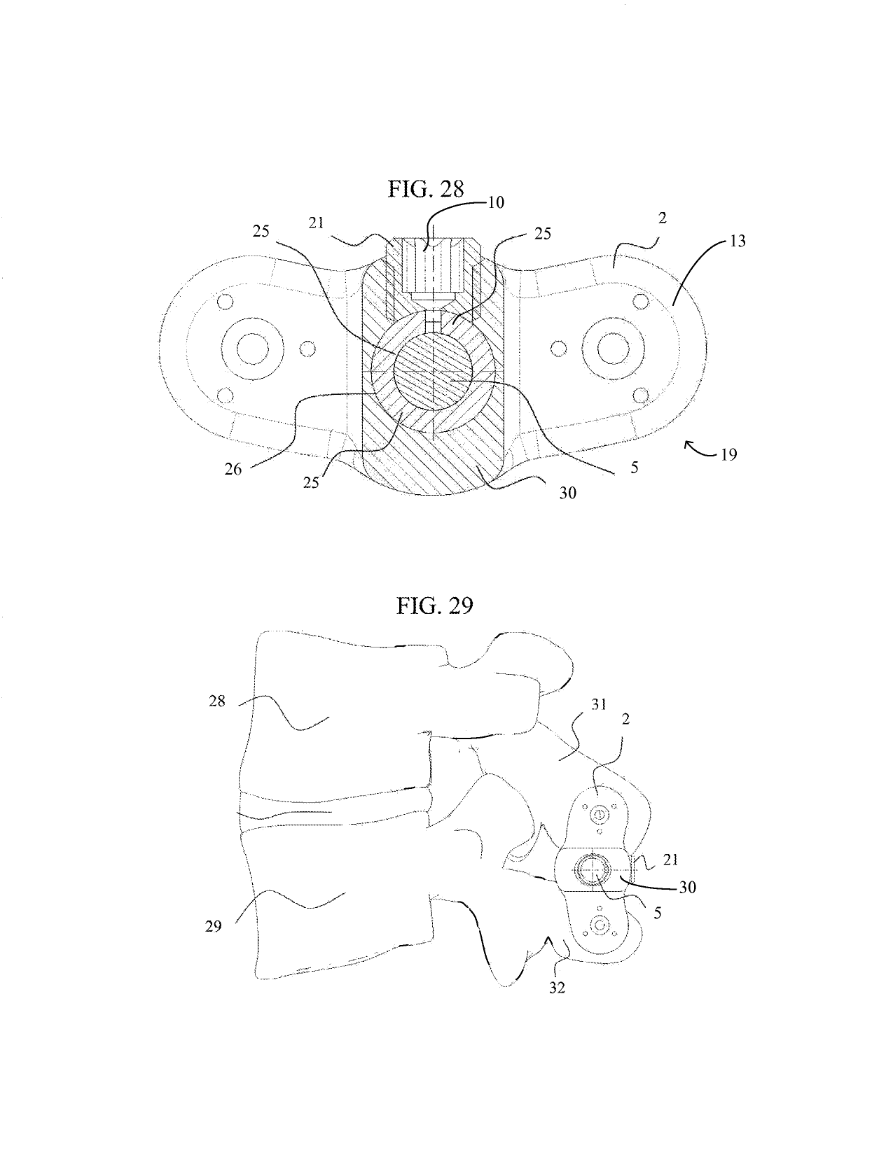

FIG. 28 illustrates the locking mechanism. The set screw 21 is rotated and applies clamping force to the bushing 25 which in turn applies clamping force to the arm. The second plate 2 can be locked in position by tightening the set screw 21 into the bushing 25, and the second plate 2 can then be unlocked by loosening the set screw 21. The bushing 25 can have a split, similar to a split washer, and the set screw 21 can be positioned to tighten on the split, but in other embodiments, the bushing 25 may form a continuous circle around the arm 5. It is also possible for the bushing 25 to have a split, and the set screw 21 can be positioned at a location on the bushing 25 other than the split.

FIG. 29 illustrates a lateral view of the spinous process device 19 implanted across a first spinous process 31 and a second spinous process 32, where the first and second spinous process 31, 32 are part of a first and second vertebra 28.

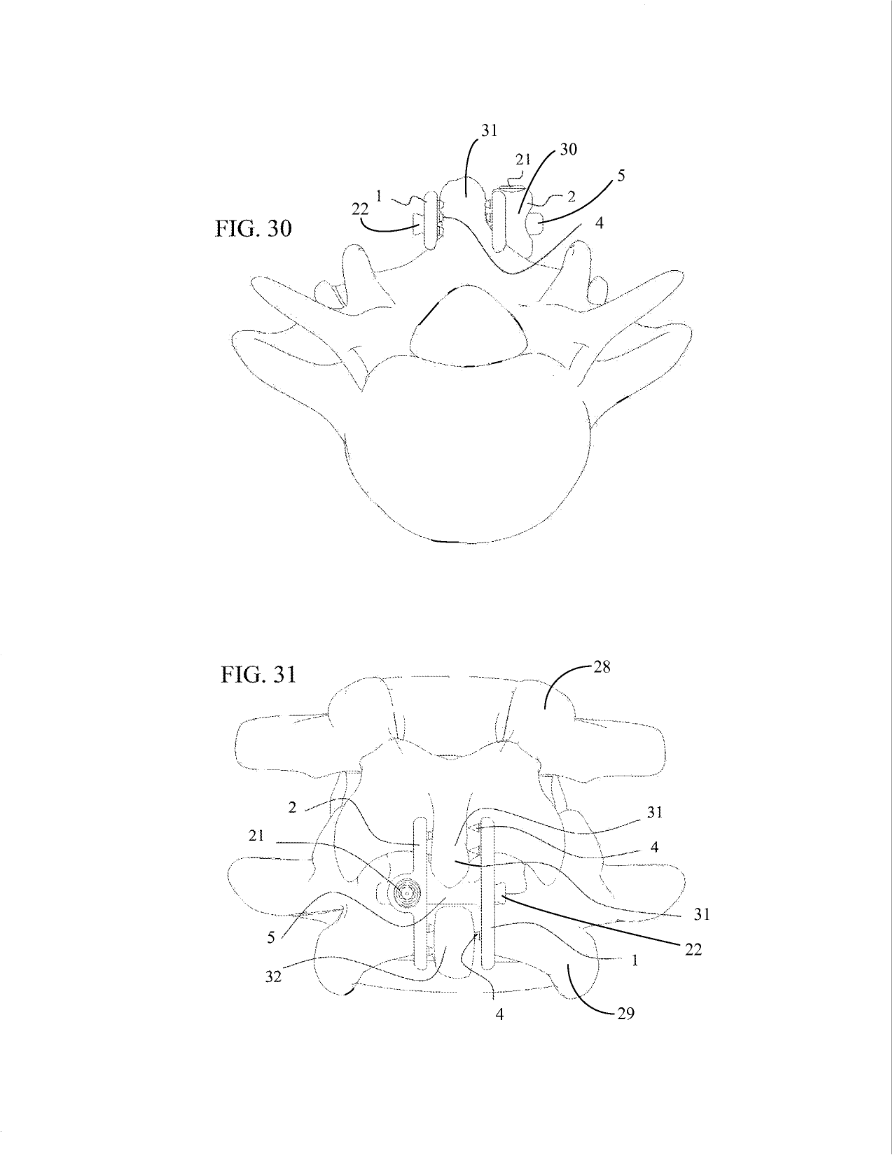

FIG. 30 illustrates an axial view of the spinous process device 19 implanted on a spinous processes 31.

FIG. 31 illustrates a posterior view of the spinous process device 19 implanted across a first and second spinous processes 31, 32. Some of the spikes 4 are shown not fully seated into the first and second spinous processes 31, 32, and the degree the spikes 4 are seated can be determined by the medical professional installing the device. Typically, the spikes 4 are at least partially seated in the spinous processes 31, 32, and possibly fully seated, when in use. The

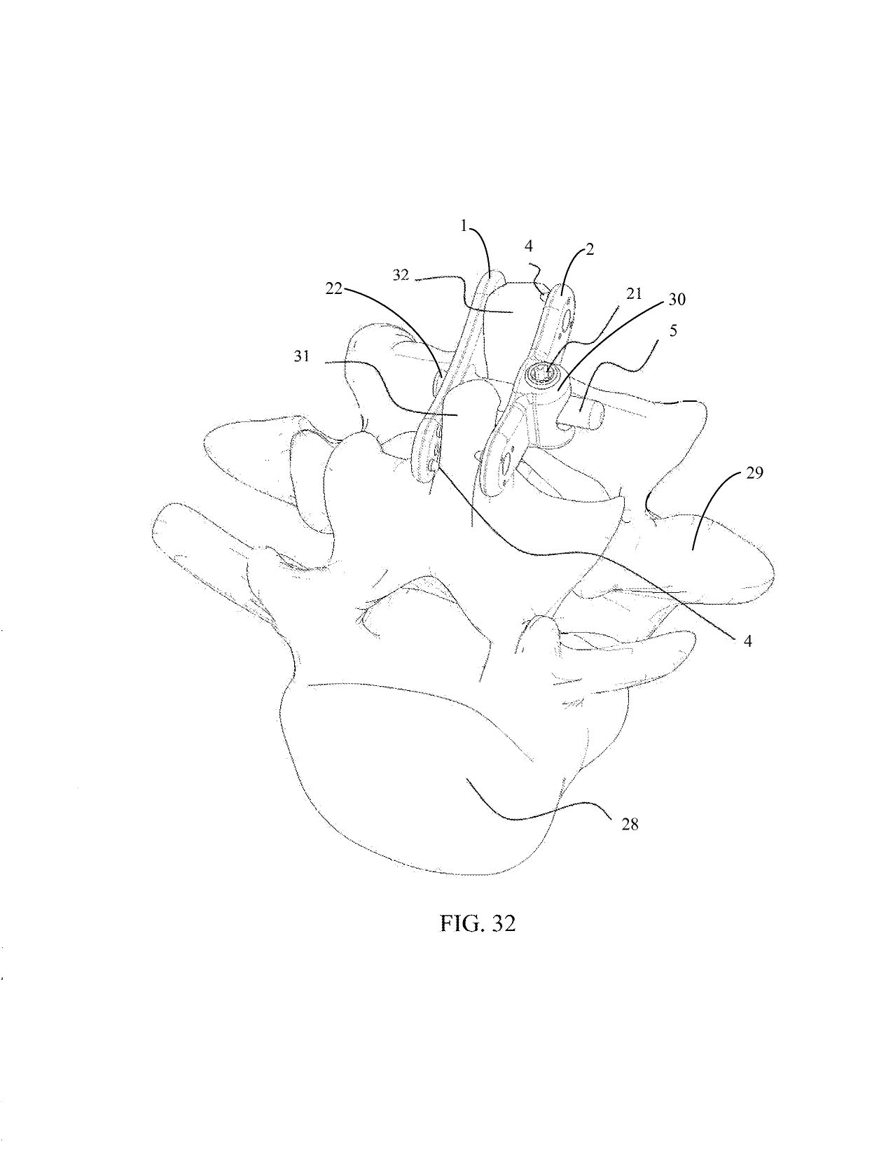

FIG. 32 illustrates a perspective view of the spinous process device 19 implanted across a first and second spinous processes 31, 32.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An implantable spinal process device comprising:
a) a first plate and a second plate, where the first and second plates further comprise a contact surface and an outer surface, and where the second plate defines a spherical cavity;
b) an arm connected to the first plate such that the arm extends from the first plate contact surface, and where the arm passes through the spherical cavity of the second plate;
c) a bushing positioned around the arm and within the spherical cavity of the second plate; and
d) a set screw penetrating the spherical cavity and contacting the bushing; and
e) where the spinal process device is sized and shaped to clamp adjacent spinal processes together.

2. The spinal process device of claim 1 where the contact surface further comprises texture for increasing friction.

3. The spinal process device of claim 2 where the texture further comprises a plurality of spikes.

4. The spinal process device of claim 1 where the bushing is split.

5. The spinal process device of claim 1 where the set screw further comprises a drive receptacle opposite a contact point, and where the contact point contacts the bushing.

6. The spinal process device of claim 1 where the arm is unitary with the first plate.

7. The spinal process device of claim 1 where the arm is rigidly fixed perpendicular to the first plate contact surface.

8. The spinal process device of claim 1 further comprising a holding tab extending from the first plate outer surface,

9. The spinal process device of claim 1 where the spherical cavity defines a contact surface opening and an outer surface opening, and where the arm extends through both the contact surface opening and the outer surface opening,

10. The spinal process device of claim 1 where the arm is cylindrical.

11. An implantable spinal process device comprising:
a) a first plate and a second plate, where the first and second plates further comprise a contact surface and an outer surface, and where the second plate defines a pocket;
b) an arm extending from the first plate contact surface, where the arm further comprises a head with a larger cross section than the arm, and where the head is sized and shaped to fit within the pocket; and
c) where the spinal process device is sized and shaped to clamp adjacent spinal process together.

12. The spinal process device of claim 11 where the contact surface comprises a friction surface.

13. The spinal process device of claim 12 where the texture comprises a plurality of spikes.

14. The spinal process device of claim 12 where the first plate contact surface further comprises a threaded socket, where the arm further comprises a threaded region opposite the head, and where the threaded region is sized and shaped to screw into the threaded socket.

15. The spinal process device of claim 14 further comprising a drive receptacle positioned in the head.

16. The spinal process device of claim 14 where the threaded socket extends perpendicular to the first plate contact surface.

17. The spinal process device of claim 1 where the head comprises angled sides, and where the pocket further comprises an interference region sized and shaped to match the angled sides of the head.

18. The spinal process device of claim 17 where the second plate defines a relief area which is an opening extending from the pocket to an edge of the second plate, and where the relief area is sized to allow passage of the arm but not the head.
